(19) United States
(12) Patent Application Publication
(10) Pub. No.: US 2004/0115251 A1
(43) Pub. Date: Jun. 17, 2004

(54) Polymerized hydrogel adhesives with low levels of monomer units in salt form

(75) Inventors: Stephen Allen Goldman, Citta Sant'Angelo (IT); Wolfgang Edgar Huhn, Chieti (IT)

Correspondence Address: THE PROCTER & GAMBLE COMPANY, INTELLECTUAL PROPERTY DIVISION, WINTON HILL TECHNICAL CENTER - BOX 161, 6110 CENTER HILL AVENUE, CINCINNATI, OH, 45224, US

(73) Assignee: The Procter & Gamble Company, Cincinnati, OH (US)

(21) Appl. No.: 10/675,283

(22) Filed: Sep. 30, 2003

Related U.S. Application Data
(63) Continuation of application No. PCT/US02/10177, filed on Mar. 29, 2002.

(30) Foreign Application Priority Data
Mar. 30, 2001 (EP) 01870071.6
Mar. 30, 2001 (EP) 01879971.7

(51) Int. Cl. A61K 9/70; A61F 5/44

(52) U.S. Cl. 424/443; 604/344

(57) ABSTRACT

The present invention relates to hydrogel adhesives for attachment to mammalian skin which exhibit particularly good attachment in excess moisture conditions, through the solution of the rheology conditions and level of monomers in salt form.

According to a specific embodiment of the present invention, hydrogel compositions are provided, which provide particularly good peel strength and cohesiveness properties; through the selection of monomer unit type of degrees of neutralization.

The present invention also encompasses personal care products containing the active adhesives, such as disposable waste management articles, disposable absorbent articles, and various functional articles for attachment to the human body.

### CROSS REFERENCE TO RELATED APPLICATION

This application is a continuation application of prior copending International Application No. PCT/US02/10177, with an international filing date of Mar. 29, 2002, and filed in English.

### FIELD OF THE INVENTION

The present invention relates to hydrogel adhesives which are capable of attaching to mammalian skin and exhibit excellent attachment and painless removal properties, including in excess moisture conditions, and which show excellent cohesion performance. The present invention also relates to personal care products containing the hydrogel adhesive herein, such as waste-management articles, absorbent articles, and a variety of functional articles to be worn by a human.

### BACKGROUND OF THE INVENTION

While hydrogel body adhesives for use in consumer products such as absorbent articles and waste-management articles have previously been described in, respectively, EP 1 025 823 and EP 1 025 866, the disclosure of hydrogel adhesive has mainly occurred in the context of medical applications, such as skin electrodes, transdermal drug delivery and wound healing. In EP 1 025 823 and EP 1 025 866, certain needs for consumer products such as absorbent and human waste-management products are disclosed, including secure attachment, painless removal and stability of adhesion in presence of excess moisture. In WO 00/46319 and WO 00/45864 are disclosed hydrogel adhesives for use in e.g. biomedical skin showing improved adhesion on wet skin and oily skin.

It has now been found that hydrogel compositions showing excellent attachment, in excess moisture conditions, can be formulated through the selection of the level of monomers units in salt form in said compositions, and rheology characteristics. Furthermore it has been found that the selection of monomers and degree of neutralization surprisingly enhance cohesiveness performance, while providing excellent peel strength performance.

### SUMMARY OF THE INVENTION

The present invention relates to hydrogel adhesives for attachment to mammalian skin comprising 10-60 wt % of a cross-linked hydrophilic polymer; 5-80 wt % of a water-soluble nonionic humectant, and from about 10-85 wt % water wherein the hydrophilic polymer comprises at least 50 mole %, preferably 80 mole %, most preferably 90 mole % of one or more weak-acid monomer units having a pKa above 3, the weak-acid monomer being at least 50 mole %, preferably at least 70 mole %, most preferably at least 85 mole %, still most preferably in the range of 85 mole % to 95 mole % in its acid form, the level of monomer in salt form in said hydrophilic polymer not exceeding 30 mole % of all monomer units, and that the elastic modulus of the hydrogel at a temperature of 25° C. G′₂₅ (1 rad/sec), is in the range of 2000 Pa to 6000 Pa, preferably 4000 to 5500 Pa.

The nonionic humectant is preferably glycerol, and the weak-acid is preferably acrylic acid.

In a specific embodiment herein, the present invention is directed to hydrogel adhesives for attachment to mammalian skin comprising 10-60 wt % of a cross-linked hydrophilic polymer, 5-80 Wt % of a water-soluble nonionic humectant and 10-85 wt % water, characterized in that the polymer comprises at least 90 mole %, preferably 95 mole % weak acid monomer units which is from 75 mole % to 95 mole %, preferably 85 mole % to 95 mole % in its acid form, and wherein G′₂₅ (1 rad/sec) is in the range of 1000 Pa to 10,000 Pa, preferably 2000 Pa to 6000 Pa, more preferably 4000 Pa to 5500 Pa, the preferred nonionic humectant being glycerol and the weak acid being acrylic acid.

The present invention also encompasses personal care products, particularly human waste management devices, absorbent articles and functional articles, comprising a hydrogel according to the embodiments described above.

### DETAILED DESCRIPTION

The hydrogel adhesives herein contain 10-60 wt % of a cross-linked hydrophilic polymer, 5-80 wt % of a water-soluble nonionic humectant, and 10-85 wt % water. The polymerization of the monomers preferably takes place in presence of the nonionic humectant and water and cross-linking creates a 3-dimensional matrix for the polymer, also referred to as gel form and hydrogel.

The hydrophilic polymer includes repeating units or monomers which contain at least 50 mole % of one of more weak-acid monomers, more preferably 80 mole %, most preferably 90 mole % of said weak-acid monomers.

Weak-Acid Monomer:

The weak acid monomer is defined in relation to its pKa, which must be above 3. The said monomers are preferably selected from the group of olefinically unsaturated carboxylic acids and carboxylic acid anhydrides such as acrylic acid, methacyclic acid, maleic acid, itaconic acid, crotonic acid, ethacrylic acid, citroconic acid, fumaric acid, β-sterylacrylic acid and the like. Particularly preferred weak-acid monomers are acrylic acid and methacrylic acid, acrylic acid being most preferred.

In addition to the nature of the monomer, the respective amounts of and neutralization forms of said monomer is particularly critical in achieving the unique properties of the present hydrogel compositions, in terms of its ability to exhibit excellent adhesive performance even in excess moisture conditions.

Indeed, it has been found that in order to achieve such performance in excess moisture condition, the level of monomers units in salt form in the hydrogel herein should not exceed 30 mole %, preferably 20 mole % of all monomer units in the polymer. The level of monomer units in salt form directly impacts the rate at which the hydrogel absorbs physiological fluid such as urine, and thus the hydrogels of the present invention have been found to exhibit a saline absorption rate of less than 2.5×10⁻³ g/cm²/sec^{0.5}, preferably less than 2.0×10⁻³ g/cm², even more preferably less that 1.5×10⁻³ g/cm²/sec^{0.5} according to the test method described hereinafter. Without being bound by theory, it is believed that the disassociation of the counterions of monomer units in salt form from the polymer decreases the osmotic driving force for hydrogel swelling and thus the driving force for absorption of physiological fluids such as urine. This decreases the rate for absorption of these fluids. By reducing the rate of absorption for fluids in contact with the hydrogel, the quantity of absorbed fluid is decreased, thus reducing the degree to which exposure to physiological fluids impacts adhesion and cohesion properties.

Saline absorption rate is measured by exposing the adhesive surface of a circular section of the hydrogel to excess saline solution under conditions where (i) the hydrogel is restrained from swelling in the lateral directions and (ii) it is allowed to swell in the z-direction (perpendicular to the plane of the hydrogel) under a confining pressure of 0.3 psi (2.07 kPa) by absorption of 0.9% saline solution. The quantity of saline solution absorbed as a function of time is measured and the saline absorption rate is calculated as described in detail below from the variation of absorbed saline versus time.

The weak-acid monomer of the present invention should therefore be present at least 50 mole % in their acid form, preferably at least 70 mole %, most preferably at least 85 mole %, most preferably from 85 to 95 mole % in acid form.

The selection of weak-acid monomers as the predominant monomer units for the hydrogel of the present invention, is driven by the following considerations:

Without being bound by theory, it is believed that decreased hydrophilicity of the hydrogel surface improves the adhesion of the hydrogel, especially to hydrophobic surfaces such as skin. It is believed that the ability of the hydrogel to spread onto and into the interstices of the skin surface is aided when the surface energy of the hydrogel is closer to the surface energy of the skin. It is believed that decreased surface hydrophilicity of the hydrogel is particularly important for less-deformable and less-flowable hydrogels having relatively higher values of storage modulus and relatively lower values of tan δ (as described below), rheological properties that are desirable for improving the cohesiveness of the hydrogel and reducing the residue left behind on the surface (e.g., skin) when the hydrogel adhesive is repositioned and/or detached after use.

It is also believed that an increased hydrophobicity of the hydrogel surface reduces the rate of absorption of water when the hydrogel is exposed to high humidity environments and increases the capability of the hydrogel to retain its adhesion properties when it does absorb some water in a high humidity environment and/or when the hydrogel comes into direct contact with physiological fluids. It is further believed that the initial contact angle of a droplet of water on the surface of the hydrogel provides a measure of the hydrophilicity of the hydrogel surface, with a higher contact angle indicating a less hydrophilic and more hydrophobic surface.

Without being bound by theory, it is believed that, for the purpose of increasing the contact angle and reducing the rate of saline absorption, the weak-acid monomers of the present invention are particularly useful; when partially neutralized; these weak-acid monomers may contribute to a high contact angle by their ability to transfer protons from acid-form weak-acid monomers within the bulk of the hydrogel to salt-form weak-acid monomers on the surface, thus decreasing the hydrophilicity of the surface. In their acid form, these weak acid monomers also do not contribute significantly to the driving force for absorption of physiological fluids as discussed hereinabove. This decrease in surface hydrophilicity and saline absorption rate is achieved without the need to excessively decrease the bulk hydrophilicity of the hydrogel (e.g. by incorporating significant concentrations of non-ionic monomers.) Such a decrease in bulk hydrophilicity can be detrimental to other desirable properties of the hydrogel, such as its compatibility to skin, its ability to maintain a favorable pH, its ability to absorb sufficient water for adhering to wet skin, its ability to absorb perspiration, etc.

Contact angle is measured by depositing a droplet of distilled water on the surface of the hydrogel and measuring the initial angle of the drop with respect to the surface using the optical method and calculational methods described hereinafter in test methods. In determining the contact angle of the hydrogel, it is important that the measurement not be influenced by the deposition of a surface layer of hydrophobic material on the hydrogel. Such a surface layer could, for example, be transferred from a siliconized release paper applied to the exposed surface of the hydrogel.

Hydrogels of this invention preferably have a contact angle of at least 40 degrees, more preferably at least 50 degrees, even more preferably at least 60 degrees, even more preferably at least 70 degrees and most preferably at least 90 degrees.

Humectant:

The 3-dimensional adhesive matrix also comprises a humectant or mixture of humectants (also referred herein as a plasticizer), which is preferably a liquid at room temperature. The humectant is selected such that the monomer and polymer may be solubilized or dispersed within. For embodiments wherein irradiation cross linking is to be carried out, the humectant is desiderably irradiation cross linking compatible such that is does not significantly inhibit the irradiation cross linking process of the polymer. The components of the humectant mixture are preferably hydrophilic and miscible with water.

Suitable humectants include alcohols, polyhydric alcohols such as glycerol and sorbitol, and glycols and ether glycol such as mono- or diethers of polyalkylene glycol, mono- or diester polyalkylene glycols, polyethylene glycols (typically up to to a molecular weight of about 600), glycolates, glycerol, sorbitan esters, esters of citric and tartaric acid, imidazoline derived amphoteric surfactants, lactams, amides, polyamides, quaternary ammonium compounds, esters such as phthalates, adipates, stearates, palmitates, sebacates, or myristates, glycerol esters, including mono/di/tri-glycerides, and combinations thereof. Particularly preferred are polyhydric alcohols, polyethylene glycol (with a molecular weight up to about 600), glycerol, sorbitol and mixtures thereof. Glycerol is especially preferred. The humectant comprises 5-80 wt % of the hydrogel.

An important function of the humectant is to reduce the water activity of the hydrogel to 0.35-0.95, preferably 0.4-0.85, most preferably from 0.45-0.75. Water activity is determined by measuring the equilibrium relative humidity above the hydrogel according to the method described hereinafter in the test methods section

Rheology:

The viscous behaviour of the adhesive can be interpreted to represent an indication of the ability of the adhesive to quickly attach and securely adhere to a particular surface. The elastic behaviour can be interpreted as an indication of the “hardness” behaviour of the adhesive. Its value is also important for good initial attachment. Their combination is believed to be an indicator of the required force upon removal. The relation between elastic and viscous modulus is considered to be an indication on which fraction of the removal energy will be dissipated within the adhesive and which fraction is available to trigger the actual removal.

In order to provide adhesives for secure initial and prolonged attachment and easy/painless removal, the relation between the elastic modulus and the viscous modulus as well as their dynamic behaviour is also of importance. While not being bound by theory, it is believed that for hydrogels applied to skin, the rheological properties at T=37° C. are most relevant to adhesion and removal properties. However, for the hydrogels of this invention, it has been found that the rheology properties are only at most moderately sensitive to temperature in the range of 25-37° C. Thus, for the purpose of this invention, it is convenient to specify the rheological properties at a temperature of 25° C. The adhesive has an elastic modulus at a temperature of 25° C. abbreviated G′₂₅, a viscous modulus at a temperature of 25° C. of G″₂₅, and the ratio of G″₂₅/G′₂₅ at 25° C., referred to as tan δ₂₅.

It has been found that, in order to perform effectively, including in excess moisture conditions, the adhesives according to the present invention must have a G′₂₅ in the range 2000-6000 Pa, most preferably in the range of 4000 to 5500 Pa.

It is also an important attribute to the composition, herein that they exhibit very good cohesiveness, to prevent residue of adhesive on the skin.

To achieve this, the tan δ₂₅ (1 rad/s) of the composition herein is preferably selected such that it lies within the range of than 0.15 to 0.65, preferably 0.15 to 0.55.

It has been determined that the relation between the thickness or caliper C, measured in millimeters (mm), of the layer in which the adhesive is provided, typically onto at least a portion of the wearer facing surface of the articles, and the viscous modulus G″₂₅ at about 100 rad/sec of the adhesive, is relevant to the scope of providing an easy and painless removal from the wearer's skin of such a adhesive applied on at least a portion of the wearer facing surface of an absorbent article for attachment of said article to the skin of a wearer.

The adhesive of the present invention is thus preferably provided as a layer having a thickness C such that the viscous modulus G″₂₅ (100 rad/sec) and the thickness C preferably satisfy the following empirical equation:

G″₂₅≦[(1.53+C)×1724]Pa

Adhesion Properties:

The hydrogels herein preferably have a 90° peel force on dry skin of between 0.3 to 3N/cm, more preferably 1.5 to 3 N/cm. Peel force can also be measured at 180° on Polyethyleneterephthalate (PET). The hydrogels herein preferably have a peel force on PET of between 0.2-5 N/cm, more preferably of between 0.5-5 N/cm. The methods for measuring peel force on skin and PET are described hereinafter in the test methods section.

Furthermore, the hydrogels herein show a particularly good adhesion performance on wet-skin and in the presence of excessive moisture, such as can be found in high-humidity applications or when directly exposed to physiological fluids. A method for measuring peel force after water absorption is disclosed hereinafter.

Preferred Hydrogels

Preferred hydrogels according to a specific embodiment of the present invention combine a tan δ₂₅ (1 rad/s) of 0.15 to 0.65, preferably 0.15 to 0.65 with a peel force on dry skin of 0.3-3 N/cm, preferably 1.5 to 3N/cm, achieving optimal cohesiveness performance; it has been found that the maintenance of both characteristics in said ranges is warranted if the level of weak-acid preferably acrylic acid in the hydrogels herein, is at least 90% mole %, preferably at least 95 mole % and said weak-acid is at least 75 mole %, preferably 85 mole % in its acid form, more preferably 85 mole % to 95 mole % in its acid form.

Accordingly such preferred hydrogels of the present invention comprises a hydrogel adhesive for attachment to mammalian skin comprising 10-60 Wt % of a cross-linked hydrophilic polymers, 5-80 Wt % of a water-soluble non-ionic humectant and 10-85% water, characterized in that the polymer comprises at least 90 mole % weak-acid monomer, preferably 95 mole % weak-acid monomer, where the weak-acid monomer is preferably acrylic acid, where the weak-acid monomer is at least 75 mole % in its acid form, preferably at least 85 mole % in its acid form, more preferably 85 mole % to 95 mole %, and wherein G′₂₅ (1 rad/sec) is in the range of 1000 Pa to 10,000 Pa, preferably 2,000-6000 Pa, more preferably 4000 Pa to 5500 Pa, the humectant being preferably glycerol.

Said hydrogels according to the embodiment herein, are preferably such that the counterion for the weak-acid monomer unit in salt form is a mono, di, or tri-valent metal ion or combination thereof. Sodium and potassium are especially preferred counterions.

Polymerization Conditions:

According to the present invention the polymer component of the adhesive can be physically, chemically or ionically cross linked in order to form the 3 dimensional matrix. Physical cross linking refers to polymers having cross links which are not chemical covalent bonds but are of a physical nature such that for example there are areas in the 3 dimensional matrix having high crystallinity or areas having a high glass transition temperature or areas having hydrophobic interactions. Chemical cross linking refers to polymers which are linked by covalent chemical bonds. The polymer can be chemically cross linked by radiation techniques such as UV-, E beam-, gamma or micro-wave radiation or, preferably by co-polymerizing the monomers with a di/poly-functional monomer crosslinker via the use e.g., of UV, thermal and/or redox polymerization initiators.

Suitable polyfunctional monomer, monomer crosslinkers include polyethyleneoxide d(meth)acrylates with varying PEG molecular weights, IRR280 (a PEG diacrylate available from UCB Chemical), trimethylolpropane ethoxylate tri(meth)acrylate with varying ethyleneoxide molecular weights, IRR210 (an alkoxylated tryacrilate: available from UCB Chemicals), trimethyolpropane tri(meth)acrylate, divinylbenzene, pentaerythritol triacrylate, pentaeythritol triallyl ether, triallyl amine, N,N-methylene-bis-acrylamide and other polyfunctional monomer crosslinkers known to the art. Preferred polyfunctional monomer crosslinkers include the polyfunctional diacrylates and triacrylates.

The monomers of the present invention are preferably polymerized via the use of a free radical polymerization initiatior. Such free-radical polymerization initiators are well known in the art and can be one or more photoinitiator(s), thermal initiator(s), or redox initiator(s) and be present in quantities up to 5% by weight, preferably from 0.02% to 2%, more preferably from 0.02% to 0.4%. Photoinitiators are preferred. Suitable photoinitiators include type I-[ ]-hydroxy-ketones and benzyldimethyl-ketals e.g. Irgacure 651 (dimethoxybenzylphenone; available from Ciba Specialty Chemicals) which are believed, on irradiation with UV frequencies, to form benzoyl radicals that initiate polymerization. Particularly preferred photoinitiators include 2-hydroxy-2-methyl-propiophenone (available under the trade name of Darocur 1173 from Ciba Specialty Chemicals), I-hydroxycyclohexylphenylketone (available under the trade name Irgacure 184 from Ciba Specialty Chemicals) and 4-(2-hydroxyethoxy)phenyl-(2-hydroxy-2-methylpropyl) ketone (available under the trade name of Irgacure 2959 from Ciba Specialty Chemicals). Suitable thermal initiators include potassium persulfate and VA044 (available from Wako). Suitable redox initiators include the combination of hydrogen peroxide and ascorbic acid and sodium persulfate and ascorbic acid.

Chemical crosslinking can also be effected after polymerization by use of polyfunctional reagents capable of reacting with polymer functional groups such as ethyleneglycol diglycidyl ether, polyols such as glycerol, and other polyfunctional reagents known to the art.

Crosslinking can also be effected all or in part by ionic crosslinking wherein groups of opposite charge interact via ionic interactions. Suitable ionic crosslinking agents include those known to the art including polyvalent cations such as Al⁺³ and Ca⁺², d/poly-amines, d/poly-quaternary ammonium compounds, including polymeric polyamines and polyquaternary ammonium compounds known to the art.

In preparing adhesive compositions in accordance with the invention, the ingredients will usually be mixed to provide a reaction mixture in the form of an initial pre-gel aqueous based liquid formulation, and this is then converted into a gel by a free radical polymerization reaction as described above. This may be achieved for example using conventional thermal initiators and/or photoinitiators or by ionizing radiation. Photoinitiation is a preferred method and will usually be applied by subjecting the pre-gel reaction mixture containing an appropriate photoinitiation agent to UV light after it has been spread or coated as a layer on siliconised release paper or other solid or porous substrate. The incident UV intensity, at a wavelength in the range from 240 to 420 nm is of sufficient intensity and exposure duration (e.g. 10-3000 mW/cm²) to complete the polymerization in a reasonable time. To facilitate the process, it is often preferable to expose the reaction mixture to several UV irradiation sources, in sequence. The processing will generally be carried out in a controlled manner involving a precisely predetermined sequence of mixing and thermal treatment or history.

The total UV irradiation time should preferably be less than 300 seconds, more preferably less than 60 seconds, and even more preferably less than 10 seconds to form a gel with better than 95% conversion of the monomers, preferably more that 99.9% of monomers, even more preferably more than 99.99% of monomers. Those skilled in the art will appreciate that the extent of irradiation will be dependent on the thickness of the reaction mixture, reactivity and concentration of the monomers, concentration of photoinitiator, properties of the humectant, and nature of substrate on to which the reaction mixture is coated and the source of UV.

While some polymer can be incorporated into the reaction premix, the incorporation of an excessive level of this pre-polymer is believed to be detrimental to forming hydrogel adhesives with the favorable combination of rheology and cohesiveness. Without being bound by theory, it is believed that this pre-polymer has a tendency to remain as a component of the sol fraction of the hydrogel and to not be effectively incorporated into the three-dimensional polymer matrix (i.e., the gel fraction of the hydrogel). Thus, it is preferred that this pre-polymer comprise less than 10 mole %, preferably less than 5 mole % of the total monomer units of the reaction pre-mix and of the polymer in the hydrogel.

These timings are for high pressure mercury arc lamps as the source of UV operating at 200 W/cm. The peak intensity of UV reaching the surface of the substrate is approximately 1000 m/W/cm². For a given lamp, the UV intensity is a function of the operating power and distance of the reaction mixture from the UV source. Also, a high-pass UV filter can be employed to minimize exposure to UV intensities of very-low wave length.

In order to minimize and preferably eliminate the presence of any residual monomers it is important to ensure that the reaction is complete. This is dependent upon a number of factors such as the substrate onto which the adhesive is applied, the type and intensity of the ultra violet light and the number of ultra violet light passes.

Optional Ingredients:

Common additives known in the art such as polymerization inhibitors, chain transfer agents, surfactants, soluble or dispersible polymers, buffers, preservatives, antioxidants, pigments, mineral fillers, and the like and mixtures thereof may also be comprised within the adhesive composition in quantities up to 10% by weight each respectively. Preferably, the hydrogels herein should contain no salt or minimum levels, below 1% by wt, preferably below 0.5% by wt.

Other suitable monomers can also be incorporated at amounts up to about 50 mole % of the polymer. These monomers can be selected from eg. strong-acid monomers: the strong acid monomer is defined in relation to its pKa, which must be below 3. The pKa is measured by tritation of the acid with strong base in acqueous solution according to methods well known in the art. The said strong acid monomers are preferably selected from the group of olefinically unsaturated aliphatic or aromatic sulfonic acids such as 2-acrylamido-2-methylpropanesulfonic acid, 3-sulphopropyl(meth)acrylate, 2-sulfoethyl(meth)acrylate, vinylsulfonic acid, styrene sulfonic acid, allyl sulfonic acid, vinyl toluene sulfonic acid, methacrilyc sulfonic acid and the like. Particularly preferred strong-acid monomers are 2-acrylamido-2-methylpropanesulfonic acid, 3-sulphopropyl(meth)acrylate, 2-sulfoethyl(methacrylate; others suitable monomers can be selected from nonionic, zwitterionic, or cationic monomers known to those skilled in the art. Examples of nonionic monomers include N,N-dimethylacrylamide, acrylamide, N-isopropyl acrylamide, hydroxyethyl(meth)acrylate, hydroxypropyl(meth)acrylate, alkyl(meth)acrylates, N-vinyl pyrrolidone and the like. Examples of cationic monomers include N,N-dimethylaminoethyl (meth)acrylate, N,N-dimethylaminoethyl(meth)acrylamide and the respective quaternary salts and the like.

pH:

The pH of the hydrogel composition herein is in the range of from 3 to 6, more preferably 3 to 5.5, most preferably from 3.5 to 5.5, which represents values perfectly compatible with the pH of mammalian skin.

This pH range is directly achievable by the compositions herein, without, without the use of any additional buffering agent, which can have a detrimental impact on the performance and skin friendliness of the hydrogels herein.

The conditions of measure of the pH are described hereinafter in the test methods section.

Personal Care Products

For the purpose of the present invention, personal care products means products, disposable or reusable, which are designed to be worn by a human in contact or close proximity to the body in order to achieve a function directed to the person's heath, well-being, comfort or pleasure.

A first type of such articles includes disposable, human waste management devices such as urine, menstrual and faecal management devices.

Disposable Waste-Management Devices

Urine, menstrual or faecal management devices herein include bags having an aperture and a flange surrounding the aperture for adhesive attachment to the uro genital area and or the perianal area of a wearer. Any faecal, menstrual or urine management device known in the art can be provided with an adhesive according to the present invention. Such devices are described for example in WO 99/00084 and WO 99/00085.

The urine, menstrual or faecal management devices herein also includes devices designed to be attached to artificial apertures in the body, such as ostomy/colostomy devices.

The bag as used in such articles is a flexible receptacle for the containment of urine, menstrual and excreted faecal matter.

The bag is designed to safely contain any entrapped material, typically it will be liquid impermeable, yet it may be breathable. The bag is designed of sufficient strength to withstand rupture in use, also when pressure on the bag is exerted in typical wearing conditions, such as sitting.

The bag may contain absorbent material. The absorbent material may comprise any absorbent material which is capable of absorbing and retaining liquids. The absorbent material may comprise a wide variety of liquid-absorbent materials commonly used in disposable diapers and other absorbent articles such as comminuted wood pulp, which is generally referred to as airfelt.

The human waste management device in particular urine/menstrual management devices according to the present invention may also comprise an additional acquisition layer. The acquisition layer is typically secured to the inner surface of bag. However, the acquisition layer may also be secured to the flange, or both the flange and the inner surface of bag. The acquisition layer is preferably positioned such that it separates the genitalia of the wearer from coming into direct contact with the absorbent material. The acquisition layer is fluid pervious allowing urine/menses to readily pass through so that it may be absorbed by absorbent material.

The bag is provided with an aperture whereby excreted matter is received from the body prior to storage within the bag cavity. The aperture is surrounded by a flange and may be provided in any shape or size, such as circular, oblong, heart shaped and may be symmetrical or asymmetrical, preferably the aperture has an oblong configuration either in the longitudinal or in the transversal direction or in both directions, e.g. the contours of the aperture are in the shape of two ellipses with the respective main axes being substantially perpendicular.

The flange comprises a garment facing surface and a wearer facing surface. In an preferred embodiment these are two large, substantially flat surfaces, however, the flange may also comprise projections designed to fit the perineal or coccygeal area of the wearer.

The flange should be made of soft, flexible and malleable material to allow easy placement of the flange to the perianal area. Typical materials include nonwoven materials, wovens, open celled thermoplastic foams, closed-cell thermoplastic foams, composites of open celled foams and stretch nonwoven, and films. A closed-cell foam of polyethylene has been found effective, but more preferably an open celled polyurethane foam is used. Preferably, such foams have a thickness within the general range of 0.1 to 5 millimetres and a basis weight of 5 to 250 g/m², more preferably 50 g/m². Other thermoplastic foam materials, or other suitable plastics sheet materials having the described properties of such foams (i.e., softness, pliability, stretchability, and contractability) might also be used.

The adhesive can be applied to the wearer facing surface of the flange by any means known in the art such as slot coating, spiral, or bead application or printing. Typically the adhesive is applied at a basis weight of from 20 g/m² to 2500 g/m², more preferably from 500 g/m² to 2000 g/m² most preferably from 700 g/m² to 1500 g/m² depending on the end use envisioned. For example, for faecal management devices to be used for babies the amount of adhesive may be less than for faecal management devices designed for active adult incontinence sufferers.

Disposable Absorbent Articles

Another type of personal care articles herein include disposable absorbent articles such as diaper, sanitary napkins, pantiliners, tampons, perspiration pads. Absorbent articles as used herein mean articles comprising an absorbent core, and can be made by any of the ways usual in the art. The application of the adhesive to the wearer facing surface, typically the topsheet surface of an absorbent article should not cause major problems to those skilled in the art since it can be provided by any well known techniques commonly used to apply adhesives. Most preferably the adhesive is provided in a pattern of small incremental areas such as dots or similar.

This invention can be used beneficially on disposable absorbent articles which are applied directly to the skin of a user. The article usually exhibits absorbency for bodily fluids, the protection of the user's garments from soiling, is comfortable to the user, and is easy to produce and to package. The disposable absorbent article is described below by reference to a sanitary napkin or catamenial, however diapers, panty liners, adult incontinence articles, tampons or perspiration pads are also included under the term disposable absorbent articles.

Other Personal Care Products

The present invention the adhesive herein may also find application to other personal care products. The adhesives may for example find utility to adhere functional articles which adhere to the skin such as cosmetic or pharmaceutical delivery articles which provide a substance to the skin such as skin treatment substances, cream, lotions, hormones, vitamins, deodorants, drugs; cosmetic or pharmaceutical delivery articles provide a substance to emanate away from the skin such as insecticides, inhalation drugs, perfumes and; functional articles which are not necessarily attached to the skin, but which require a high residence time on the skin such as decorative cosmetics, (lipstick, eye shadow, stage make-up) and cleaning articles (hand cleaners, face masks and hygienic pore cleansers). Such articles are preferably non-absorbent for bodily liquids.

The adhesive may also in addition find application to attach articles to the skin such as protective articles such as genital-, knee- or elbow-protectors or bandages; clothing such as bras, surgical gowns, or parts of garments during fitting at a tailor; nasal plasters; prosthesis such as breast replacements or wigs; cold wraps e.g. to provide pain relief from bruises and to reduce swelling; thermal wraps comprising thermal cells as disclosed for example in WO 97/36968 and WO 97/49361 to provide relief of temporary and chronic pain such as neck wraps as disclosed in for example U.S. Pat. No. 5,728,146, knee wraps exemplified in WO 97/01311, and back wraps as disclosed for example in U.S. Pat. No. 5,741,318; hearing aids; protective face masks (for the reduction or prevention of inhalation of noxious substances); anti-snoring patches, ornamental articles such as jewelry, earrings, guises, tattoos; goggles or other eye wear, tapes, bandages, dressings of general utility, wound healing and wound management devices; and biomedical skin electrodes such as ECG, EMG, EEG, TENS electrosurgery, defibrillation, EMS and electrodes for facial/beauty applications; and fixation products and/or devices intended to affix patient catheters, tubing leadwires cables etc.

Test Methods

1. Rheology

The rheology of hydrogels is measured at 25° C. using a RHEOMETRICS SR 5000 oscillatory rheometer or the equivalent. A sample of thickness of approximately 1 mm and diameter of 25 mm is placed between two insulated Parallel Plates of 25 mm diameter, controlled at a temperature of approximately 25° C. using a Peltier system or equivalent. A Dynamic Frequency Sweep is performed on the hydrogel in either stress or strain mode at an applied strain within the linear elastic response of the hydrogel (e.g., up to a strain of about 10%), with measurements at discrete frequency values between 0.1 and 100 rad/sec. Results are quoted as G′, G″ and tan delta at frequency values of 1.0 and 100 rad/sec. The hydrogel is aged at least 24 hours before measurement. The average of at least three determinations are reported.

2. Peel Force on Dry Skin

The peel force to remove hydrogel from dry skin is measured using a suitable tensile tester, for example an Instron Model 6021, equipped with a 10N load cell and an anvil rigid plate such as the Instron accessory model A50L2R-100. Samples are cut into strips of width 25.4 mm and length between about 10 and 20 cm. A non-stretchable film of length longer than the hydrogel is applied to the reverse side of the hydrogel sample (e.g., the substrate side) using double sided adhesive. A suitable film is 23μ thick PET, available from Effegidi S.p.A, 43052, Colorno, Italy. For samples with release paper, the release paper is removed prior to applying the hydrogel to the forearm and then rolling it into place using a compression weight roller to prevent air entrapment between hydrogel and skin. The roller is 13 cm in diameter, 4.5 cm wide and has a mass of 5 Kg. It is covered in rubber of 0.5 mm thickness. The free end of the backing film is attached to the upper clamp of the tensile tester and the arm is placed below. The sample is peeled from the skin at an angle of 90 degrees and a rate of 1000 mm/min. The average peel value obtained during peeling of the whole sample is quoted as the peel value in N/cm. The average of triplicate measurements is reported.

3. Peel Force on PET

Peel force to remove hydrogel from poly(ethylene teraphthalate) (PET) film is measured using a suitable tensile tester, for example an Instron Model 6021, equipped with a 10N load cell and attachment for a rigid lower plate, e.g. steel, oriented along the direction of cross-head movement. Freshly produced hydrogel is stored in a closed aluminium bag or similar for at least 12 to 24 hours at room temperature before measuring. A defect free sample of at least 10 cm in length is cut from the hydrogel sample. A piece of double sided adhesive, for example type 1524 from 3M Italia S.p.A, I-20090 Segrate, Italy, at least 130 mm long and 25.4 mm wide is stuck to the back side of the hydrogel laminate. The hydrogel is cut along the tape's outer edges. The second liner is removed from the tape and it is stuck on the rigid base plate. A strip of standard PET of 23μ thickness and no corona treatment, is cut to about 300 mm×40 mm. Suitable material would include “Cavilen-Forex” from Effegidi S.p.A, Via Provinciale per Sacca 55, 1-43052 Colorno, Italy. The release liner is removed from the hydrogel and the bottom end fixed to the rigid plate by regular tape. The standard substrate is then applied onto the body adhesive using a hand roller once forward and once backward at a speed of 1000 to 5000 mm/min. The roller is 13 cm in diameter, 4.5 cm wide and has a mass of 5 Kg. It is covered in rubber of 0.5 mm thickness. The measurement is preferably performed within 10 minutes of application of the substrate.

The free end of the standard substrate is doubled back at an angle of 180 degrees and the rigid plate is clamped in the lower clamp of the tensile tester. The free end of the standard substrate is fixed in the upper clamp of the tensile tester. The peel test is performed at a speed of 1000 mm/min. The initial 20 mm of peel is disregarded and the average force over the remaining length is quoted as the peel force in N/cm. The average of triplicate measurements is reported.

4. Peel Force After Water Absorption

To determine the effect of water gain on the peel force of the hydrogel from skin, a sample of hydrogel polymerized on a suitable substrate is pre-treated such that a pre-determined weight percent of water is gained by the hydrogel, prior to measurement as described in the peel force on dry skin or peel force on PET test method sections. Moderate water gains are accomplished by placing a known weight of hydrogel in an environmental chamber, for example, a Termaks model PHDO-02 or equivalent, at about 38° C. and 85% relative humidity and weighing at intervals until the required weight gain is achieved. For larger water gains, a known weight of water can be distributed uniformly onto the surface of the hydrogel and allowed to be absorbed. On reaching the required weight addition of water, the sample is stored in a vapor-tight bag to equilibrate for at least 24 hours, preferably at least 48 hours, before measuring the peel force as previously described. A comparable untreated control sample of the hydrogel is measured as a reference. The average of triplicate determinations for both test and control samples are reported. The percentage change in peel force for the test sample versus the control sample is also reported:

% Change=100*(Peel Force_{water gain}−Peel Force_{control})/Peel Force_{control}

5. Initial Contact Angle

The contact angle of a sessile drop of water on hydrogel film is measured using, for example, a Krüss DSAIO (or equivalent) instrument. Immediately after deposition on the surface of the hydrogel of a drop of purified water (HPLC grade or equivalent) of approximately 10 ul volume successive images of the drop are captured electronically and approximated to the best drop shape by a dedicated software, for example, Krüss Drop Shape Analyser software. About 25 frames per second are recorded for the first 2-3 seconds. Frames acquired before drop stabilization are discarded, then the relationship between contact angle and time is extrapolated, via linear extrapolation, to Time=0 to give the “initial contact angle” of water on hydrogel; an average of at least 3, preferably at least 5 measurements, are taken.

6. pH

The pH of the hydrogel is measured using an electronic pH meter, for example as supplied by Mettler Toledo, and a flat bulb electrode, for example type InLab 426, calibrated as per the manufacturers instructions. The bulb is brought into contact with the surface of the gel and the measurement is recorded after some seconds, once the value on the display is constant. The electrode is rinsed with distilled water between successive measurements.

7. Water Activity (Relative Humidity)

Relative humidity is measured using an electronic humidity probe, for example the Testo 650 supplied by Testo GmbH & Company, calibrated as per the manufacturers instructions. A sample of hydrogel is placed inside the measuring chamber and sealed. Measurements are preferably made at approximately 25° C. The relative humidity and temperature are displayed on the instrument and recorded when constant. This is typically between about 30 minutes and several hours. The water activity is the relative humidity divided by 100.

8. Vapor-Phase Absorption

This measurement is applicable to hydrogel samples with initial water activity (as measured using the method described herein) of less than 0.85. A sample of hydrogel having a basis weight of approximately 1 kg/m² is used for this measurement. If not prepared with a vapor-impervious backing, a vapor-impervious backing (e.g., PET) is attached to the underside of a hydrogel sample. The hydrogel sample is positioned exposed-size up on an electronic balance that is pre-positioned inside a humidity and temperature controlled chamber, capable of controlling the humidity to ±2% and temperature to ±1C, for example, a Termaks model PHDO-02 or equivalent), that has been pre-equilibrated at 85% RH and 38° C. The initial weight of the hydrogel sample is recorded as well as the change in weight of the sample as a function of time. The weight of the sample is monitored for at least 16 hours or until the sample weight has reached equilibrium. The percentage weight gain of the hydrogel at equilibrium is calculated from the recorded weight gain at equilibrium and the initial weight of the hydrogel sample, after the combined weight of any substrate or film is subtracted from the weight of the hydrogel sample. The weight gain as a function of time in units of g/cm² is calculated from the weight gain as a function of time and the calculated area of the hydrogel sample. This weight gain is fitted to the function:

W(t)=W_{f}*(1−exp(−kᵥt))

where W(t) is the weight gain of the hydrogel at time t, W_{f} is the weight gain of the hydrogel at equilibrium, and kᵥ is a rate constant in units of sec⁻¹ that describes the kinetics of water-vapor absorption. The average values from at least duplicate determinations for kᵥ and the percentage weight gain at equilibrium are reported.

9. Saline Absorption Rate

A sample of hydrogel having a basis weight of approximately 1 kg/m² is used for this measurement. A demand absorbency apparatus (as described in detail in Goldman et. al. U.S. Pat. No. 5,599,335) filled with a 0.9 wt % saline solution is used to measure the kinetics of saline solution absorption by the hydrogel. The fritted funnel of the demand-absorbency apparatus is prepared for measurement by rinsing with saline, draining excess fluid, drying, positioning at a fixed height of ˜2 mm above the top surface of the fluid reservoir, and sequentially: (i) positioning the valve connecting the fritted funnel so that it is open to the fluid reservoir, (ii) positioning the valve connecting the fritted funnel so that it is open to a drain tube of approximately −5 cm hydrostatic suction for a time period of ˜5 minutes, and (iii) positioning the valve connecting the fritted funnel so that it is isolated from both the reservoir and the drain tube. A piston/cylinder apparatus as described in U.S. Pat. No. 5,599,335 is used to confine the hydrogel during the demand absorbency measurement. The cylinder has an inner diameter of 60 mm and a cylinder bottom permeable to saline but impermeable to hydrogel. A stainless steel weight is positioned on top of the piston such that the combined weight of the piston and weight is equivalent to 0.30 psi (2.07 kPa). From a larger piece of the hydrogel, a punch (or equivalent) is used to obtain a cylindrical section of hydrogel with diameter between 57-60 mm. This hydrogel is centrally positioned inside the cylinder with adhesive surface facing the cylinder bottom and the piston positioned such that the hydrogel is confined between the cylinder bottom and the piston. A variety of approaches can be used to position the hydrogel in the piston/cylinder apparatus such that there is minimal contact between hydrogel and the walls of the cylinder during insertion including: (i) adhering the hydrogel to the piston before inserting the piston and hydrogel into the cylinder, (ii) resting the substrate surface of a hydrogel sample on the inverted piston and slipping the inverted cylinder over the piston and hydrogel, etc. The piston/cylinder apparatus is then centered on the fritted disk of the fritted funnel, the stainless steel weight is inserted into the piston, the fritted funnel cover is positioned onto the funnel, and the experiment is initiated by positioning the valve connecting the fritted funnel so that it is open to the fluid reservoir. The quantity of saline absorbed by the hydrogel is measured as a function of time for a time period of at least one hour. The weight gain between times of 100 and 3600 seconds is fitted to the function:

W(t)=(kₛt^{0.5})+c

where W(t) is the weight gain of the hydrogel (in grams) per unit area of the cylinder (28.27 cm²) in units of g/cm² and kₛ is a rate constant in units of g/cm²/sec^{0.5} that describes the kinetics of saline absorption. Weight gain data at times shorter than 100 seconds is excluded from this fit due to a variety of factors that can bias and/or otherwise impact the initial absorption rate. The average slope for at least two determinations is reported as the saline absorption rate.

### EXAMPLES

The first part of the experiments below focuses on the stability of hydrogel in excess moisture conditions, corresponding to the main embodiment of the invention herein.

The following compositions were prepared:

### Example 1

Approximately 31.96 parts of acrylic acid (BASF) are slowly dissolved, with stirring in approximately 25.02 parts of water. To the cooled solution is slowly added, with stirring and cooling, approximately 3.55 parts of 50% sodium hydroxide (NaOH; Aldrich), which is sufficient to convert approximately 10 mole % of the acrylic acid to sodium acrylate. During this addition the temperature is maintained below 25° C. The mixture is stirred for several minutes to allow any precipitated sodium acrylate to redissolve. After dissolution, approximately 39.48 parts of glycerol (Agrar) are added. The final premix is stirred until a complete solution is accomplished. The resultant solution is covered to shield it from light. The composition of this solution, including the water produced via the neutralization of the acid monomers, is given in Table 1.

Aliquots of the above monomer solution are polymerized to form a hydrogel. Prior to polymerization, approximately 0.177 parts of the polyfunctional crosslinker IRR210 (a polyoxyethylene triacrylate crosslinker from UCB) and 0.228 parts of Darocur 1173 (2-hydroxy-2-methyl-propiophenone; Aldrich) is added to approximately 100 parts of the monomer solution and dispersed and/or dissolved with stirring for at least 15 minutes.

One fraction of the monomer solution is extruded at a basis weight of approximately 1.0 kilograms per square meter onto a thin, porous non-woven substrate (for example, 911 NW available from Fuller; non woven basis weight of 27 gsm). The monomer solution is polymerized via UV irradiation curing. The peak power density and the total energy density of the lamps are measured using an UV Power Puck (E.I.T. Inc.) and the output intensity and energy in the UV-A range of the lamps are adjusted so that the incident UVA peak power density on the sample is approximately 1,100 Watt/cm² and the UVA energy density is approximately 18.2 J/cm² (measured with the UV filter). The sample is passed at the line speed of 5 meter per minute underneath twelve consecutive lamps equipped with UV filters (for example Bte Bedampfungstechnik GmbH filters, with Transmittance (T)=50% at 320 nm, T<1% in the range 220-310 nm, T>85% in the range 330-2000 nm) to polymerize the monomer solutions and convert them into adhesive hydrogels. After polymerization a release liner (for example CS42 form Cogesil) is applied to the hydrogel and it is rolled up for storage.

A second fraction of the monomer solution is spread at a basis weight of approximately 1.0 kilograms per square meter onto siliconized release paper (for example, CO.GE.SIL “Silkraft-70g” (Palazzo)), that has been surface treated by wiping with a very-thin layer of Pluronic 6400 surfactant (BASF) to facilitate spreading of the solution. For handling purposes, the release paper is pre-positioned inside a 8.5 cm diameter polystyrene Petri dish. The UV irradiation equipment, procedure and conditions described above are then used to effect polymerization. The above procedure is repeated to obtain several samples of hydrogel polymerized on release paper.

The resultant hydrogels are analyzed according to the test methods described above. From samples polymerized on release paper, a 25 mm diameter punch is used to obtain a circular sample of hydrogel for measurement of rheology properties. Rheology measurements are made for at least three samples punched from separately-polymerized hydrogel samples on release paper. The average thickness of the sample is obtained from the plate-to-plate separation used for the rheology measurement.

Results:

As can be seen from the results in Table 2, G′ (1 rad/s) is between 4000-5500 Pa, and the value for saline absorption rate of less than 1.5*10⁻³ g/cm²/sec^{0.5}. The composition of Example 1, having the Rheology and monomer salt-form values according to the present invention, thus proved to have excellent performance as regard its stability in excess moisture conditions; said performance was confirmed by the following experiment.

To samples of the hydrogel of Example 1 polymerized on non-woven substrate having pre-determined weights, distilled water in pre-determined quantities were added uniformly over the surface. These samples were then sealed in a vapor-tight bag and equilibrated at ambient temperature for at least five days. After equilibration, the hydrogel samples were removed from the bag and analyzed gravimetrically for water gain. The hydrogel samples were then analyzed for peel on dry skin—all on the same individual—using the procedure described in the test methods section. The results are given in Table 3. The results show that water gain up to about 20 wt % has minimal impact on peel on dry skin for the hydrogel of Example 1.

### Example 2 (Comparative)

An approximately 58 wt % aqueous solution of 2-acrylamido-2-methyl-1-propanesulphonic acid-monosodium salt (NaAMPS) is prepared by adding approximately 52.4 parts of 2-acrylamido-2-methyl-propanesulphonic acid (Lubrizol or equivalent) to approximately 27.3 parts of an aqueous solution containing approximately 0.5 parts of phosphate buffer (KH2PO4; Aldrich), for pH control, and approximately 100 ppm of 4-methoxy-phenol (Aldrich), an inhibitor to prevent premature polymerization. To this mixture is slowly added, with stirring, approximately 20.2 parts of 50% sodium hydroxide (NaOH; Aldrich). During this addition the temperature is maintained below 35° C. Addition of NaOH is continued until the pH of the solution increases to approximately 5.0. The final solution is cooled to ambient temperature.

Approximately 22.3 parts of the NaAMPS solution and approximately 19.2 parts of acrylic acid is added to approximately 13.3 parts of distilled water. To this solution is added, with stirring and cooling, approximately 6.4 parts of 50% NaOH (Aldrich), which is sufficient to convert approximately 30 mole % of the acrylic acid to sodium acrylate. During this addition the temperature is maintained below 35° C. After the resultant solution is cooled to ambient temperature, approximately 38.9 parts of glycerol (Agrar) is added with stirring. The resultant solution is covered to shield it from light. The composition of this solution, including the water produced via the neutralization of the acid monomers, is given in Table 1. Prior to polymerization, approximately 0.15 parts of IRR210 and 0.228 parts of Darocur 1173 are added to approximately 100 parts of the monomer solution and dispersed and/or dissolved with stirring for at least 15 minutes.

One fraction of the monomer solution is spread at a basis weight of approximately 1.0 kilograms per square meter onto siliconized release paper for example, CO.GE.SIL “Silkraft-70g” (Palazzo), that has been surface treated by wiping with a very-thin layer of Pluronic 6400 surfactant (BASF) to facilitate spreading of the solution. For handling purposes, the release paper is pre-positioned inside a 8.5 cm diameter polystyrene Petri dish. A second fraction of the monomer solution is coated at a basis weight of approximately 1.0 kilogram per square meter onto a thin, porous non-woven substrate (Fiberweb 33; Corolind PE; 33 g/sqm). This nonwoven is backed by a PET film (Cavilen—Forex; 23 μm), which is attached to the non-woven by 3M 1524 double-sided adhesive. This non-woven is pre-positioned inside a suitable container, for example a 12 cm by 12 cm polystyrene Petri-dish. The solution is added dropwise over the surface of the non-woven and then spread by gently tilting the box from side-to-side. An IST Model # M20-1(2)-TR-SLC UV Polymer Reactor, equipped with an IST 200 ozone-free arc lamp (Spectrum Type: CKII-OF) is used to effect polymerization. A high-pass UV filter with a frequency cut-off of approximately 310 nM (UV Filter Borofloat T320 from Bedamfungs-technik) is positioned between the lamp and the sample to filter out low-frequency UV irradiation The monomer-coated substrate is irradiated while passing underneath the lamp on a variable-speed belt positioned approximately 13 cm underneath the lamp. The speed of the belt is set at approximately 5 meter/min. The peak output power of the lamp is measured using an UMD-1 power meter (Eta Plus Electronic) and the output intensity of the lamp is adjusted so that the incident peak UV power on the sample is approximately 1000 milliwatt/cm². Twelve consecutive passes of the sample underneath the lamp is used to polymerize the monomer solution and convert it into a soft adhesive hydrogel. The above procedure is repeated to obtain several samples of hydrogel on each type of substrate.

The resultant hydrogels are analyzed as described in Example 1. The results are summarized in Table 2. For this hydrogel, the level of monomer units in salt form is approximately 42 mole %, and this resulted in a value for saline absorption rate (kₛ) of greater than 2.5*10⁻³ g/cm²/sec^{0.5}.

The second part of the experimental work is directed to the peel strength/cohesion performance, according to another aspect of the present invention.

The following compositions were prepared:

### Example 3

The procedure described in Example 1 for preparing the monomer solution is followed except that approximately 32.07 parts of acrylic acid (BASF) are dissolved in approximately 20.96 parts of water. Approximately 1.78 parts of 50% NaOH is added, which is sufficient to convert approximately 5 mole % of the acrylic acid to sodium acrylate. Approximately 45.19 parts of glycerol are added. The composition of this solution, including the water produced via the neutralization of the acid monomers, is given in Table 4. Prior to polymerization, approximately 0.2295 parts of IRR210 and 0.2280 parts of Darocur 1173 are added to approximately 100 parts of the monomer solution.

One fraction of the monomer solution is spread at a basis weight of approximately 1.0 kilograms per square meter onto siliconized release paper for example, CO.GE.SIL “Silkraft-70g” (Palazzo), that has been surface treated by wiping with a very-thin layer of Pluronic 6400 surfactant (BASF) to facilitate spreading of the solution. For handling purposes, the release paper is pre-positioned inside a 8.5 cm diameter polystyrene Petri dish. A second fraction of the monomer solution is coated at a basis weight of approximately 1.0 kilogram per square meter onto a thin, porous non-woven substrate (for example 911 NW, available from Fuller, NW basis weight of 27 gsm). This non-woven is pre-positioned inside a suitable container, for example a 12 cm by 12 cm polystyrene Petri-dish. The solution is added dropwise over the surface of the non-woven and then spread by gently tilting the box from side-to-side. The UV irradiation is then used to effect polymerization. The above procedure is repeated to obtain several samples of hydrogel on each substrate. The resultant hydrogels are analyzed as descrived in Example 1 for rheology measurements; to measure peel strength, several 10-12 cm by 2.54 cm strips are cut from one or more hydrogel samples formed on the nonwoven substrate. These strips are used to measure the peel of the adhesive from PET and from skin, using the methods described above.

As can be seen from the results in Table 4, tan δ (1 rad/s) is less than 0.55 resulting in very good cohesiveness results; measured by visual inspection of residues on skin. Neutralizing the acrylic acid to 5 mole % results in a high peel force for attachment to dry skin and PET.

### Example 4

The procedure descrived in Example 3 is followed except that approximately 28.84 parts of acrylic acid are dissolved in approximately 18.28 parts of water. Approximately 6.49 parts of 50% NaOH is added, which is sufficient to convert approximately 20 mole % of the acrylic acid to sodium acrylate. Approximately 46.52 parts of glycerol are added. The composition of this solution, including the water produced via the neutralization of the acid monomers, is given in Table 4. Prior to polymerization, approximately 0.1765 parts of IRR210 and 0.2280 parts of Darocur 1173 are added to approximately 100 parts of the monomer solution.

The resultant hydrogels are analyzed as described in Example 3. The results are summarized in Table 5. As can be seen from the results, tan δ (1 rad/s) is less than 0.65. Neutralizing the acrylic acid to 20% results in a relatively high peel force for attachment to dry skin.

### Example 5

The procedure described in Example 3 is followed except that approximately 24.88 parts of acrylic acid are dissolved in approximately 13.54 parts of water. Approximately 13.82 parts of 50% NaOH is added, which is sufficient to convert approximately 50 mole % of the acrylic acid to sodium acrylate. Approximately 47.84 parts of glycerol are added. The composition of this solution, including the water produced via the neutralization of the acid monomers, is given in Table 4. Prior to polymerization, approximately 0.1765 parts of IRR210 and 0.2280 parts of Darocur 1173 are added to approximately 100 parts of the monomer solution.

The resultant hydrogels are analyzed as described in Example 3. The results are summarized in Table 5. As can be seen from the results, tan δ (1 rad/s) is less than 0.65 resulting in a hydrogel with good cohesion; however, neutralizing the acrylic acid to 50% results in a lower peel force compared to Example 4 for attachment to both dry skin and PET.

TABLE 1
Composition of Monomer Solutions

| | | | | Monomers | | | | |
|---|---|---|---|---|---|---|---|---|
| | Acrylic | Na | | in | | | | |
| | Acid | Acrylate | NaAMPS | Salt | | | Daracur | |
| Hydrogel | (moles/ | (moles/ | (moles/ | Form | Water | Glycerol | 1173 | IRR210 |
| (Ex #) | kg) | kg) | kg) | (mole %) | (wt %) | (wt %) | (wt %) | (wt %) |
| 1 | 3.98 | 0.44 | 0 | 10 | 27.6 | 39.5 | 0.23 | 0.18 |
| 2 | 1.86 | 0.80 | 0.56 | 42 | 27.2 | 38.9 | 0.23 | 0.15 |

TABLE 2
Test Results For Example 1 & 2 Hydrogels

| | | | | G′ | G″ | | |
|---|---|---|---|---|---|---|---|
| | G′ | G″ | | (25° C.) | (25° C.) | | |
| Hydrogel | (25° C.) | (25° C.) | Tan δ | (Pa; | (Pa; | Tan δ | kₛ * 10⁵ |
| (Ex #) | (Pa; 1 rad/s) | (Pa; 1 rad/s) | (1 rad/s) | 100 rad/s) | 100 rad/s) | (100 rad/s) | (g/cm²/s^{0.5}) |
| 1 | 5260 | 3205 | 0.61 | 23900 | 11800 | 0.49 | 1.3 |
| 2 | 4410 | 2290 | 0.52 | 17200 | 9550 | 0.55 | 2.6 |

TABLE 3
Test Results for Example 1 Hydrogel - Water Addition

| Water | | |
|---|---|---|
| Additino | Peel on Dry Skin | Change in Peel |
| (wt %) | (N/cm) | (%) |
| 0 | 1.86 | 0 |
| 8 | 1.82 | −2 |
| 11 | 1.85 | −1 |
| 18 | 1.74 | −6 |
| 23 | 1.23 | −33 |

TABLE 4
Composition of Monomer Solutions

| | Acrylic | Na | Na | | | Daracur | |
|---|---|---|---|---|---|---|---|
| Hydrogel | Acid | Acrylate | Acrylate | Water | Glycerol | 1173 | IRR210 |
| (Ex #) | (moles/kg) | (moles/kg) | (mole %) | (wt %) | (wt %) | (wt %) | (wt %) |
| 3 | 4.23 | 0.22 | 5 | 22.3 | 45.2 | 0.23 | 0.23 |
| 4 | 3.20 | 0.81 | 20 | 23.0 | 46.5 | 0.23 | 0.18 |
| 5 | 1.73 | 1.73 | 50 | 23.6 | 47.8 | 0.23 | 0.18 |

TABLE 5
Test Results for Example 3-5 Hydrogels

| | | | | | | | Peel | Peel | |
|---|---|---|---|---|---|---|---|---|---|
| | G′ | G″ | | G′ | G″ | | Force | Force | |
| Hydrogel | (25° C.) | (25° C.) | Tan δ | (25° C.) | (25° C.) | Tan δ | PET | Skin | |
| (Ex #) | (Pa; 1 rad/s) | (Pa; 1 rad/s) | (1 rad/s) | (Pa; 100 rad/s) | (Pa; 100 rad/s) | (100 rad/s) | (N/cm) | (N/cm) | Cohesivness |
| 3 | 4533 | 2452 | 0.540 | 20032 | 10933 | 0.5458 | 4.1 | 2.4 | Very good |
| 4 | 4356 | 2686 | 0.620 | 20380 | 11086 | 0.5440 | 2.6 | 1.9 | Good |
| 5 | 4295 | 2535 | 0.590 | 19656 | 12073 | 0.6142 | 1.2 | 1.2 | Good |

All documents cited in the Detailed Description of the Invention are, in relevant part, incorporated herein by reference; the citation of any document is not to be construed as an admission that it is prior art with respect to the present invention.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A hydrogel adhesive comprising from 10 weight percent to 60 weight percent of a cross-linked hydrophilic polymer, from 5 weight percent to 80 weight percent of a water-soluble non-ionic humectant, and from 10 weight percent to 85 weight percent water, wherein said hydrophilic polymer comprises at least 50 mole percent of at least one weak-acid monomer unit having a pKa above 3, said weak-acid monomer unit being at least 50 mole percent in their acidic form, said hydrophilic polymer further comprising less than 30 mole percent of all monomer units in salt form; and, wherein said hydrogel adhesive has an elastic modulus at a temperature of 25° C., G′₂₅ (1 rad/sec), ranging from 2000 Pa to 6000 Pa.

2. The hydrogel adhesive of claim 1, wherein said weak acid monomer units are present at levels of at least 80 mole percent.

3. The hydrogel adhesive of claim 1, wherein said weak-acid monomer unit is selected from the group consisting of acrylic acid, methacrylic acid, maleic acid, itaconic acid, crotonic acid, ethacrylic acid, citroconic acid, fumaric acid, β-sterylacrylic acid and combinations thereof.

4. The hydrogel adhesive of claim 1, wherein said weak acid monomer unit is present in an acidic form of at least 70 mole percent.

5. The hydrogel adhesive of claim 1, wherein said water-soluble nonionic humectant comprises a polyhydric alcohol.

6. The hydrogel adhesive of claim 5, wherein said water-soluble nonionic humectant further comprises glycerol.

7. The hydrogel adhesive of claim 1 wherein said hydrogel has a Saline Absorption Rate of less than 2.5×10⁻³ g/cm²/sec^{0.5}.

8. The hydrogel adhesive of claim 1, wherein said hydrogel adhesive has a peel strength on dry skin ranging from 0.3 N/cm to 3.0 N/cm and a viscous modulus at a temperature of 25° C., G″₂₅, wherein the ratio of G″₂₅ (1 rad/sec)/G′₂₅ (1 rad/sec) ranges from 0.15 to 0.65.

9. The hydrogel adhesive of claim 1, wherein said hydrophilic polymer comprises at least 90 mole percent of a weak acid monomer unit, said weak acid monomer unit being at least 85 mole percent in an acidic form.

10. The hydrogel adhesive of claim 1, wherein said hydrogel adhesive is capable of attachment to mammalian skin.

11. The hydrogel adhesive of claim 1, wherein said adhesive is disposed on a wearer facing surface of a disposable human waste management device, said device comprising a bag, said bag further comprising an aperture and a flange disposed about said aperture, said flange further comprising said wearer facing surface and a garment facing surface.

12. The hydrogel adhesive of claim 1, wherein said adhesive is disposed on a wearer facing surface of an absorbent article, said article comprising said wearer facing surface and a garment facing surface.

13. The hydrogel adhesive of claim 1, wherein said adhesive is disposed on a functional article, said functional article being selected from the group consisting of cosmetic delivery articles, pharmaceutical delivery articles, decorative cosmetic articles, cleaning articles, protective articles, clothing, prosthesis, cold wraps, thermal wraps, hearing aids, ornamental articles, goggles, eye wear, for attachment to the skin, and combinations thereof.

14. A hydrogel adhesive suitable for attachment to mammalian skin, said hydrogel adhesive comprising from 10 weight percent to 60 weight percent of a cross-linked hydrophilic polymer, from 5 weight percent to 80 weight percent of a water-soluble non-ionic humectant, and from 10 weight percent to 85 weight percent water, wherein said hydrophilic polymer comprises at least 90 mole percent of weak acid monomer units, said weak acid monomer units being from 75 mole percent to 95 mole percent in an acidic form; and wherein said hydrogel adhesive has an elastic modulus at a temperature of 25° C., G′₂₅ (1 rad/sec), ranging from 1,000 Pa to 10,000 Pa.

15. The hydrogel adhesive of claim 14, wherein said hydrophilic polymer comprises at least 95 mole percent weak acid monomer units, said weak acid monomer units being from 85 mole percent to 95 mole percent in an acidic form, and wherein said G′₂₅ (1 rad/sec) ranges from 4000 Pa to 5500 Pa.

16. The hydrogel adhesive of claim 14 wherein said water-soluble non-ionic humectant comprises glycerol and said weak acid monomer units comprises acrylic acid.

17. The hydrogel adhesive of claim 16 wherein said acrylic acid comprises salt form monomer units, said acrylic acid monomer units further comprising a counter ion selected from the group consisting of monovalent metal ions, divalent metal ions, trivalent metal ions, and combinations thereof.

18. The hydrogel adhesive of claim 14, wherein said hydrogel adhesive has a peel strength on dry skin ranging from 0.3 N/cm to 3.0 N/cm, and a viscous modulus at a temperature of 25° C., G″₂₅ (1 rad/sec), wherein the ratio G″₂₅ (1 rad/sec)/G′₂₅ (1 rad/sec) ranges from 0.15 to 0.65.
